(19) United States
(12) Patent Application Publication
(10) Pub. No.: US 2012/0175398 A1
(43) Pub. Date: Jul. 12, 2012

(54) STAPLING APPARATUS AND METHODS OF ASSEMBLING OR OPERATING THE SAME

(75) Inventors: William J. Sandborn, La Jolla, CA (US); Elizabeth Rajan, Rochester, MN (US)

(73) Assignee: Mayo Foundation for Medical Education and Research, Rochester, MN (US)

(21) Appl. No.: 13/301,294

(22) Filed: Nov. 21, 2011

Related U.S. Application Data
(60) Provisional application No. 61/415,990, filed on Nov. 22, 2010.

(51) Int. Cl. A61B 17/068; B23P 17/04

(52) U.S. Cl. 227/175.1; 29/428

(57) ABSTRACT

Stapling apparatus and methods of assembling or operating the stapling apparatus are described herein. The stapling apparatus described herein can be delivered through an overtube or other large channel. The stapling apparatus may, in various embodiments, allow for secure closure of, e.g., endoluminal openings such as, e.g., perforations, intentional viscerotomy (e.g., NOTES/full thickness resection), and other indications.

### RELATED APPLICATION

This application claims the benefit under 35 U.S.C. §119(e) of U.S. Provisional Patent Application No. 61/415,990, filed Nov. 22, 2010 and titled STAPLING APPARATUS AND METHODS OF ASSEMBLING OR OPERATING THE SAME, which is hereby incorporated by reference in its entirety.

### BACKGROUND

Closure of endoluminal openings such as, e.g., perforations, intentional viscerectomy (e.g., NOTES/full thickness resection), and other indications that include anastomoses and gastric remodeling is sometimes required.

Existing techniques for effecting such closures include clips glues, tissue welding, and suturing (which is difficult to achieve endoscopically). Endoluminal endoscopic stapling in many of these instances has, however, proven to be technically difficult due to the size and/or configuration of existing stapling apparatus.

Examples of some known stapling devices and fundamentals of tissue stapling are described in, e.g., U.S. Pat. Nos. 5,258,008; 7,175,648; 6,843,403; 7,695,485; 6,443,973; 6,131,789; and 7,407,077; as well as in US Patent Publication No. US 2002/0099389. Additional discussions regarding stapling may be found in Komanapalli et al., “Computer assisted surgical stapling,” Multimedia Manual of Cardiothoracic Surgery (2008).

### SUMMARY

Stapling apparatus and methods of assembling or operating the stapling apparatus are described herein. The stapling apparatus described herein can be delivered through an overtube or other large channel.

The stapling apparatus described herein may, in various embodiments, allow for secure closure of, e.g., endoluminal openings such as, e.g., perforations, intentional viscerotomy (e.g., NOTES/full thickness resection), and other indications that include anastomoses and gastric remodeling.

In a first aspect, some embodiments of the medical stapling apparatus described herein may include: a first stapling module and a second stapling module, wherein each of the first and second stapling modules comprises: a first jaw comprising a first end and a second end; a second jaw comprising a first end and a second end, wherein the first end of the second jaw is pivotally attached to the first end of the first jaw, wherein the second jaw comprises a staple retaining structure adapted to retain one or more staples on the second jaw; an actuating mechanism capable of selectively rotating the first jaw and the second jaw between an open position and a closed position, wherein the second ends of the first jaw and the second jaw are closer to each other in the closed position than in the open position; and a guide lumen extending through the first jaw and/or the second jaw of the stapling module.

In some embodiments of the first aspect of the medical stapling apparatus described herein, the second ends of the first and second jaws of the first and second stapling modules comprise complementary alignment elements such that the second ends of the first and second stapling modules align with each other when the second ends of the jaws of the first stapling module face the second ends of the jaws of the second stapling module. In some embodiments, the complementary alignment elements comprise magnetic alignment elements and/or mechanically interlocking structures.

In some embodiments of the first aspect of the medical stapling apparatus described herein, the apparatus further comprises a pushing element capable of pushing the first stapling module and/or the second stapling module along a guide located in the guide lumen of the stapling module.

In some embodiments of the first aspect of the medical stapling apparatus described herein, the apparatus may further include a first pushing element capable of pushing the first stapling module along a guide located in the guide lumen of the first stapling module; and a second pushing element capable of pushing the second stapling module along a guide located in the guide lumen of the second stapling module.

In some embodiments of the first aspect of the medical stapling apparatus described herein, the guide lumen in the first and/or second stapling module generally extends between the first end and the second end of the jaw such that movement of the stapling module along a guide in the guide lumen aligns the jaw with the guide.

In a second aspect, various embodiments of a method of assembling a medical stapling apparatus of the first aspect may be described herein, wherein the method may include advancing the first stapling module through a delivery lumen in a delivery device until the first stapling module exits from the delivery lumen at a distal opening, wherein advancing the first stapling module further comprises advancing the first stapling module along a guide passing through the guide lumen of the first stapling module, and wherein the first stapling module is oriented during the advancing with the second ends of the first and second jaws located ahead of the first ends of the first stapling module such that the second ends of the first and second jaws exit the delivery lumen before the first ends of the first and second jaws; advancing the second stapling module through the delivery lumen until the second stapling module exits from the delivery lumen at the distal opening, wherein advancing the second stapling module further comprises advancing the second stapling module along a guide passing through the guide lumen of the second stapling module; and aligning the first and second stapling modules with each other after the first stapling module has exited from the distal opening of the delivery lumen, wherein the aligning comprises aligning the second ends of the first and second jaws of the first stapling module with the second ends of the first and second jaws of the second stapling module.

In some embodiments of the methods of the second aspect, the first stapling module and the second stapling module are advanced over a guide.

In some embodiments of the methods of the second aspect, aligning the first and second stapling modules comprises aligning magnetic alignment elements on the first and second stapling modules.

In some embodiments of the methods of the second aspect, aligning the first and second stapling modules comprises aligning mechanically interlocking structures.

In some embodiments of the methods of the second aspect, advancing the first stapling module comprises pushing the first stapling module through the delivery lumen.

In some embodiments of the methods of the second aspect, advancing the first stapling module comprises pushing the first stapling module through the delivery lumen and wherein advancing the second stapling module comprises pushing the second stapling module through the delivery lumen. In some embodiments, pushing the first stapling module and the pushing the second stapling module are performed using the same pushing element. In some embodiments, pushing the first stapling module and the pushing the second stapling module are performed using two or more different pushing elements.

In a third aspect, some embodiments of the medical stapling apparatus described herein may include: a base stapling module that comprises: a first jaw comprising a first end and a second end; a second jaw comprising a first end and a second end, wherein the first end of the second jaw is pivotally attached to the first end of the first jaw, wherein the second jaw comprises a staple retaining structure adapted to retain one or more staples on the second jaw; an actuation mechanism capable of selectively rotating the first jaw and the second jaw between an open position and a closed position, wherein the second ends of the first jaw and the second jaw are closer to each other in the closed position than in the open position; a first guide lumen extending through the first jaw of the base stapling module; and a second guide lumen extending through the second jaw of the base stapling module. The apparatus may further include a delivery device comprising a delivery lumen that extends through the delivery device, wherein the delivery lumen terminates at a distal opening, and wherein the delivery lumen is sized to allow passage of the base stapling module through the delivery lumen; a first guide extending through the delivery device, wherein the first guide passes through the first guide lumen in the first jaw of the base stapling module; a second guide extending through the delivery device, wherein the second guide passes through the second guide lumen in the second jaw of the base stapling module; and one or more supplemental stapling modules. Each of the supplemental stapling module comprises: an anvil element comprising a first end, a second end, and a guide lumen extending through the anvil element, wherein the first guide passes through the guide lumen of the anvil element of the supplemental stapling module; and a staple driver comprising a first end, a second end, and a guide lumen extending through the staple driver, wherein the second guide passes through the guide lumen of the staple driver, and wherein the staple driver comprises a staple retaining structure adapted to retain one or more staples on the staple driver.

In some embodiments of the third aspect of the medical stapling apparatus described herein, the second end of the first jaw of the base stapling module and the second end of the anvil element comprise complementary alignment elements such that the second ends of the first jaw and the anvil element align with each other when the second end of the first jaw faces the second end of the anvil element, and wherein the second end of the second jaw of the base stapling module and the second end of the staple driver comprise complementary alignment elements such that the second ends of the second jaw and the staple driver align with each other when the second end of the second jaw faces the second end of the staple driver. In some embodiments, the complementary alignment elements comprise magnetic alignment elements and/or mechanically interlocking structures.

In some embodiments of the third aspect of the medical stapling apparatus described herein, the apparatus further comprises a pushing element capable of pushing the base stapling module through the delivery lumen of the delivery device.

In some embodiments of the third aspect of the medical stapling apparatus described herein, the apparatus further comprises: a first pushing element capable of pushing the base stapling module through the delivery lumen of the delivery device; a second pushing element capable of pushing the anvil element through the delivery lumen of the delivery device along the first guide; and a third pushing element capable of pushing the staple driver through the delivery lumen of the delivery device along the second guide.

In some embodiments of the third aspect of the medical stapling apparatus described herein, the guide lumen in the anvil element generally extends between the first end and the second end of the anvil element such that movement of the anvil element along the first guide aligns the anvil element with the first guide, and wherein the guide lumen in the staple driver generally extends between the first end and the second end of the staple driver such that movement of the staple driver along the second guide aligns the staple driver with the second guide.

In a fourth aspect, various embodiments of a method of assembling a medical stapling apparatus of the third aspect may be described herein, wherein the method may include: advancing the base stapling module through the delivery lumen in the delivery device until the base stapling module exits from the delivery lumen at the distal opening; advancing the anvil element through the delivery lumen and along the first guide passing through the guide lumen of the anvil element, wherein the anvil element is advanced until the anvil element exits the delivery lumen and the second end of the anvil element approaches the second end of the first jaw of the base stapling module; aligning and operably attaching the second end of the first jaw to the second end of the anvil element after the anvil element approaches the first jaw as it advances along the first guide; advancing the staple driver through the delivery lumen and along the second guide passing through the guide lumen of the staple driver, wherein the staple driver is advanced until the staple driver exits the delivery lumen and the second end of the staple driver approaches the second end of the second jaw of the base stapling element; and aligning and operably attaching the second end of the second jaw to the second end of the staple driver after the staple driver approaches the second jaw as it advances along the second guide.

In some embodiments of the methods of the fourth aspect, advancing the base stapling module further comprises advancing the base stapling module along the first guide and the second guide.

In some embodiments of the methods of the fourth aspect, aligning the first jaw and the anvil element comprises aligning a magnetic element on the first jaw with a complementary magnetic element on the anvil element.

In some embodiments of the methods of the fourth aspect, aligning the second jaw of the base stapling module with the staple driver comprises aligning mechanically interlocking structures on the second jaw and the staple driver.

In some embodiments of the methods of the fourth aspect, advancing the base stapling module comprises pushing the base stapling module through the delivery lumen.

In some embodiments of the methods of the fourth aspect, advancing the anvil element comprises pushing the anvil element through the delivery lumen and wherein advancing the staple driver comprises pushing the staple driver through the delivery lumen. In some embodiments, pushing the anvil element and pushing the staple driver are performed using the same pushing element. In some embodiments, pushing the anvil element and pushing the staple driver are performed using two or more different pushing elements.

In a fifth aspect, some embodiments of the medical stapling apparatus described herein may include an elongated body comprising a proximal end and a distal end and a stapling module located at the distal end of the elongated body. The stapling module may include: a first jaw comprising a proximal end and a distal end; a second jaw comprising a proximal end and a distal end; wherein the proximal end of at least one of the first jaw and the second jaw is pivotally attached to the distal end of the elongated body; staple retaining structure adapted to retain one or more staples on at least one of the first jaw and the second jaw; a tension element comprising a proximal end and a distal end, wherein the distal end of the tension element is fixedly attached to the first jaw proximate the distal end of the first jaw, and wherein the tension element extends through an opening proximate the distal end of the second jaw before extending proximally towards the proximal end of the elongated body; and a tensioning mechanism proximate the proximal end of the elongate body, wherein the proximal end of the tension element is operably attached to the tensioning mechanism, wherein actuation of the tensioning mechanism places the tension element in tension between the distal ends of the first jaw and the second jaw.

In some embodiments of the fifth aspect of the medical stapling apparatus described herein, the tension element extends through a lumen in the first jaw.

In some embodiments of the fifth aspect of the medical stapling apparatus described herein, the tension element comprises a wire.

In some embodiments of the fifth aspect of the medical stapling apparatus described herein, both the first jaw and the second jaw are pivotally attached at their proximal ends to the elongated body.

In a sixth aspect, some embodiments of methods of operating a medical stapling apparatus according to the fifth aspect described herein may include placing the tension element in tension between the distal ends of the first jaw and the second jaw, wherein the tension element moves through the opening in the second jaw when placing the tension element in tension.

The above summary is not intended to describe each embodiment or every implementation of the stapling apparatus and methods described herein. Rather, a more complete understanding of the invention will become apparent and appreciated by reference to the following Description of Illustrative Embodiments and claims in view of the accompanying figures of the drawing.

### BRIEF DESCRIPTION OF THE VIEWS OF THE DRAWING

FIG. 1 depicts one embodiment of a stapling apparatus including two opposed stapling modules, wherein the stapling modules are in an open position.

FIG. 1A is a cross-sectional view of a jaw of one stapling module.

FIG. 2 depicts the stapling apparatus of FIG. 1, with the stapling modules in the closed position.

FIG. 3 depicts one embodiment of another stapling apparatus that includes a base stapling module and one or more supplemental stapling modules advanced through a delivery device and assembled on the end of the base stapling module.

FIG. 4 depicts the stapling apparatus at an intermediate point in the assembly process.

FIG. 5 depicts an alternative embodiment of the stapling apparatus of FIGS. 3 and 4 in which the supplemental stapling modules are delivered with the anvil element and the staple driver attached to each other.

FIG. 6 depicts an alternative embodiment of the stapling apparatus of FIGS. 3 and 4 including an optional alignment element 380 and locking pin 384.

FIG. 7A depicts another embodiment of a stapling apparatus.

FIG. 7B is an enlarged view of the stapling module at the distal end of the stapling apparatus of FIG. 7A.

FIG. 8 is an enlarged view of one alternative embodiment of a stapling module that could be used at the distal end of the stapling apparatus of FIG. 7A.

FIGS. 9A and 9B depict one illustrative embodiment of a stapler-delivered device.

FIG. 9C depicts one illustrative embodiment of a auxiliary component that can be connected to the stapler-delivered device of FIGS. 9A-9B.

FIG. 9D depicts the auxiliary component attached to the stapler-delivered device of FIGS. 9A-9B.

FIG. 10 depicts a staple adapted to use in attaching an auxiliary component at a selected location.

### DESCRIPTION OF ILLUSTRATIVE EMBODIMENTS

In the following description of illustrative embodiments, reference is made to the accompanying figures of the drawing which form a part hereof, and in which are shown, by way of illustration, specific embodiments of the stapling apparatus described herein. It is to be understood that other embodiments may be utilized and structural changes may be made without departing from the scope of the present invention.

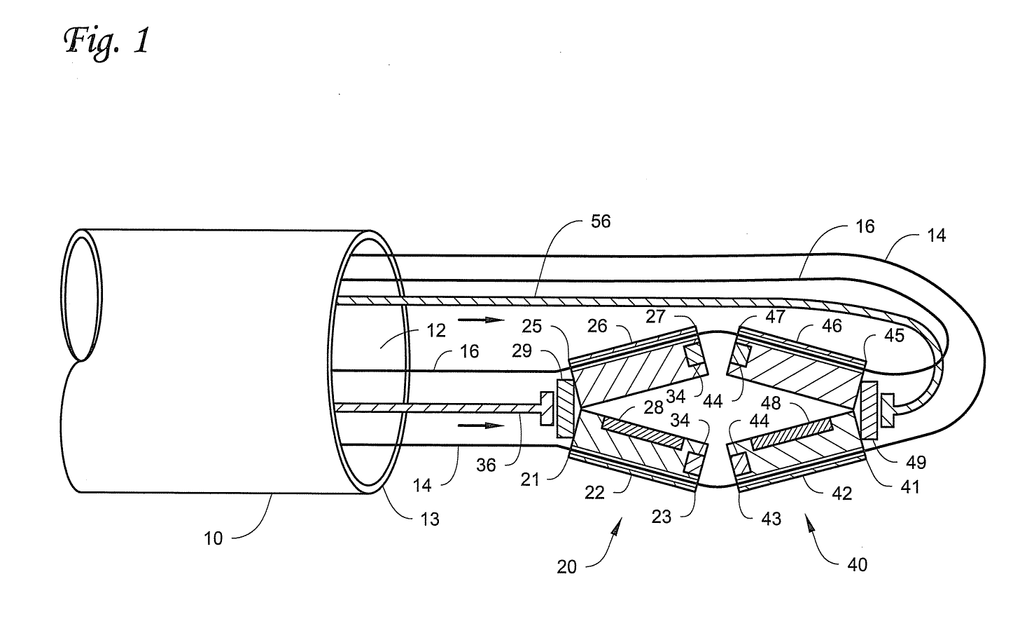

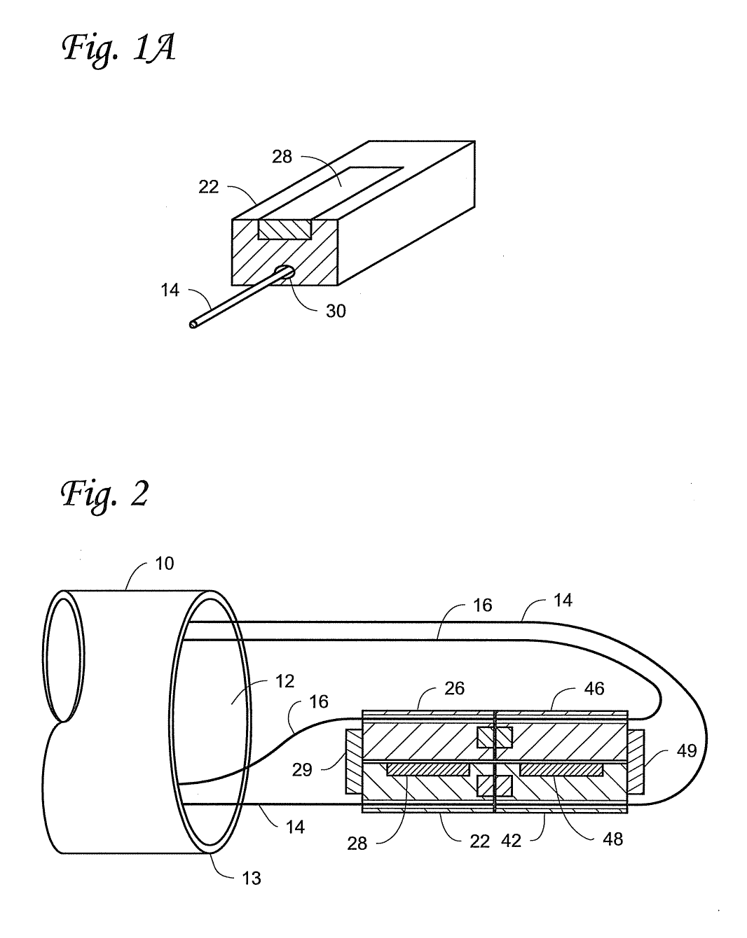

One illustrative embodiment of a medical stapling apparatus is depicted in FIGS. 1 and 2. In the depicted embodiment, the apparatus may optionally be delivered to a selected location through one or more lumens of a delivery device. The depicted delivery device 10 may be in the form of any structure that provides a lumen large enough to allow for passage of the stapling apparatus as described herein. For example, the delivery device 10 may be in the form of an overtube or other large channel structure that could, in some embodiments, be delivered using another delivery device, e.g., a colonoscope. In some embodiments, a MEGACHANNEL (a device marketed by Minos Medical, Inc. which may include, e.g., light and camera functionality) could be delivered to a selected location (e.g., a location in the colon, etc.) to deliver the delivery device described herein. In other embodiments, the delivery device may be delivered to the selected location using, e.g., a self-propelling mechanism such as, e.g., a SOFTSCOPE (Softscope Technologies, Minnetonka, Minn.). In still other embodiments, an AER-O-SCOPE (available from G.I. View Ltd) could potentially be used. These and other potentially suitable devices may be described in one or more of the following patent documents: WO 2009/032407; US 2008/0045790; and U.S. Pat. No. 6,971,990.

Although the depicted embodiment of the delivery device 10 includes a single delivery lumen 12, the various components of the stapling apparatus depicted in connection with FIGS. 1 and 2 could be delivered through different lumens in the same delivery device or even, in some embodiments, through different delivery devices.

In some embodiments, the use of different lumens in the delivery devices used to deliver the stapling apparatus described herein may enhance control and/or stability over the delivery, assembly, disassembly, and/or operation of the stapling apparatus described herein. Any such lumens provided to enhance stability and/or control may not necessarily extend the entire length of the delivery device. For example, a lumen may be located only over a distal portion of the delivery device (near the distal end 13 of the delivery device 10.

In still other embodiments, the delivery device 10 may be provided with clamping or guide structures in the interior of the delivery lumen 12, with the clamping or guide structures being useful to clamp or guide a component (e.g., a pushing element, stapling module, etc.) proximate the distal end of the delivery device 10.

The stapling apparatus depicted in FIGS. 1 and 2 includes a first stapling module 20 and a second stapling module 40. The first stapling module 20 includes a first jaw 22 and a second jaw 26. The first jaw 22 includes a first end 21 and a second end 23, while the second jaw 26 includes a first end 25 and a second end 27. The first end 21 of the first jaw 22 is pivotally attached to the first end 25 of the second jaw 26.

In the depicted embodiment, the second jaw 26 also includes staple retaining structure 28 capable of retaining one or more staples on the second jaw in a manner that allows the staple(s) to penetrate tissue located between the first jaw 22 and the second jaw 26 as described herein. The staple retaining structure 28 may take the form of, e.g., slots, openings, cassettes, etc. that retain the staple(s) before delivery to tissue, but that release the staple(s) after the staple(s) have been attached to tissue. Examples of some potentially suitable staple retaining structures may be found in, e.g., U.S. Pat. No. 5,258,008 (Wilk); U.S. Pat. No. 5,170,925 (Madden et al.), U.S. Pat. No. 5,376,095 (Ortiz); U.S. Pat. No. 5,562,241 (Knodel et al.); U.S. Pat. No. 6,488,196 (Fenton); U.S. Pat. No. 6,619,529 (Green et al.); U.S. Pat. No. 6,843,403 (Whitman); U.S. Pat. No. 7,308,998 (Mastri et al.); U.S. Pat. No. 7,743,960 (Whitman et al.); etc.

The first stapling module 20 also includes an actuation mechanism 29 that is provided to selectively rotate the first jaw 22 and the second jaw 26 between an open position (as seen in FIG. 1) and a closed position (as seen in FIG. 2). As depicted in FIGS. 1 and 2, the second ends 23, 27 of the first jaw 22 and the second jaw 26 are closer to each other in the closed position than in the open position. The actuation mechanism 29 may, in some embodiments, be electrically actuated using electrical energy delivered by any conductive elements as described in some of the stapler patents identified herein. That rotation may, in some embodiments, be accomplished with a force that is sufficient to drive any staples located in the jaws into tissue located between the jaws 22 and 26. In other embodiments, the closure and staple firing may be accomplished by separate mechanisms, systems, etc. Further examples of some potentially useful stapler mechanisms may be found in the stapler patents identified above, as well as in, e.g., U.S. Pat. No. 6,533,157 (Whitman) and U.S. Patent Application Publication US 2008/0277449 (Marczyk).

Like the first stapling module 20, the second stapling module 40 includes a first jaw 42 and a second jaw 46. The first jaw 42 includes a first end 41 and a second end 43, while the second jaw 46 includes a first end 45 and a second end 47. The first end 41 of the first jaw 42 is pivotally attached to the first end 45 of the second jaw 46.

In the depicted embodiment, the second jaw 46 also includes staple retaining structure 48 capable of retaining one or more staples on the second jaw in a manner that allows the staple(s) to penetrate tissue located between the first jaw 42 and the second jaw 46 as described herein. The staple retaining structure 48 may take the form of, e.g., slots, openings, cassettes, etc. that retain the staple(s) before delivery to tissue, but that release the staple(s) after the staple(s) have been attached to tissue. Examples of some potentially suitable staple retaining structures may be found in the stapler patent documents identified herein.

The second stapling module 40 also includes an actuation mechanism 49 that is provided to selectively rotate the first jaw 42 and the second jaw 46 between an open position (as seen in FIG. 1) and a closed position (as seen in FIG. 2). As depicted in FIGS. 1 and 2, the second ends 43, 47 of the first jaw 42 and the second jaw 46 are closer to each other in the closed position than in the open position. The actuation mechanism 49 may, in some embodiments, be electrically actuated using electrical energy delivered by any conductive elements as described in some of the stapler patents identified herein. That rotation may, in some embodiments, be accomplished with a force that is sufficient to drive any staples located in the jaws into tissue located between the jaws 42 and 46. In other embodiments, the closure and staple firing may be accomplished by separate mechanisms, systems, etc. Further examples of some potentially useful stapler mechanisms may be found in the stapler patents identified herein.

Among the features included in the embodiment depicted in FIGS. 1 and 2 are guide lumens extending through the first jaw and/or the second jaw of the stapling modules. As seen in the cross-sectional view of FIG. 1A (in which the cross-section is taken transverse to the length of the guide 14), the first stapling module 20 includes a guide lumen 30 extending through first jaw 22. A similar guide lumen can be found in the second jaw 26 of the first stapling module 20, as well as in each of the first jaw 42 and the second jaw 46 of the second stapling module 40. In some embodiments, however, only the first jaws 22, 42 or the second jaws 26, 46 of the two stapling modules 20 and 40 may include a guide lumen as described herein. The guide lumens in the first and/or second stapling modules 20, 40 may generally extend between the first ends and the second ends of the jaws such that movement of the stapling module along a guide passing through the guide lumen aligns the jaw (and, thus, the stapling module) with the guide. Furthermore, the guide lumen 30 may take the form of a channel completely enclosed within and passing through the body of the jaw 22, the guide lumens may take any form that can function to retain a guide as the jaw or other structure is advanced over the guide.

The guide lumens in the different jaws on the stapling modules 20, 40 provide structures that can be used in connection with guides 14 and 16 to deliver the stapling modules 20, 40 through the delivery device 10 and assemble them after they have exited from the distal opening 13 of the delivery lumen 12. The guides 14 and 16 may preferably be in the form of guidewires, although any structure (e.g., suture material, cables, ribbons, etc.) could be used in connection with the advancement and assembly of the stapling modules 20 and 40.

In some embodiments of the stapling modules described herein, the second ends 23, 27, 43, 47 of the first and second jaws 22, 26, 42, 46 of the first and second stapling modules 20, 40 may include complementary alignment elements 34, 44 to assist with alignment between the second ends 23, 27, 43, 47 of the first and second stapling modules 20, 40 when the second ends 23, 27 of the jaws 22, 26 of the first stapling module 20 face the second ends 43, 47 of the jaws 42, 46 of the second stapling module 40.

In some embodiments, the complementary alignment elements 34, 44 may be in the form of magnetic alignment elements that, through magnetic attraction and/or repulsion assist in aligning the second ends 23, 27 of the jaws 22, 26 of the first stapling module 20 with the second ends 43, 47 of the jaws 42, 46 of the second stapling module 40. In some embodiments, the complementary alignment elements 34, 44 may be in the form of mechanically interlocking structures, e.g., mating protrusions and recesses, keyed structures, etc. that may assist in aligning the second ends 23, 27 of the jaws 22, 26 of the first stapling module 20 with the second ends 43, 47 of the jaws 42, 46 of the second stapling module 40. In still other embodiments, both magnetic and mechanically interlocking structures may be used together to assist in alignment of the second ends 23, 27 of the jaws 22, 26 of the first stapling module 20 with the second ends 43, 47 of the jaws 42, 46 of the second stapling module 40.

Another optional feature of the stapling apparatus described herein that is provided in connection with the embodiment depicted in FIGS. 1 and 2 are the pushing elements 36, 56 that may be used to advance the stapling modules 20 and 40 through the delivery device 10. In some embodiments, one or both of the pushing elements 36, 56 may include a guide lumen through which guides 14, 16 may pass to assist in guiding the pushing elements 36, 56. The pushing elements 36, 56 may be permanently or removably attached to the stapling modules 20, 40.

One or both of the pushing elements 36, 56 may, in some embodiments, have sufficient structure that they can be steered such that the direction and/or orientation of the stapling modules 20, 40 can be controlled to assist in assembling the stapling modules 20 and 40 at a selected location after the stapling modules 20, 40 have exited from a delivery device 10. Examples of some potentially suitable devices that may be used as steerable pushing elements may be described in, e.g., the following patent documents: WO 2009/099464 (Kassab et al.); US 2004/0243227 (Starksen et al.); US2009/0171161 (Ewers et al.); etc.

By using a steerable pushing element, the first stapling module 20 can be delivered toward the selected location in an antegrade direction, while the second stapling module 40 may, upon exiting the delivery device 10, be re-oriented such that it approaches the selected location (and the first stapling module 20) in a retrograde direction—see, e.g., FIG. 1.

As discussed herein, both the first stapling module 20 and the second stapling module 40 preferably include an actuation mechanism 25, 45 that may be electrically actuated using electrical energy delivered by any conductive elements used in the stapling apparatus. For example, in some embodiments, the guides 14, 16, pushing elements 36, 56, etc. may be used to deliver electrical energy needed to move the jaws of the stapling modules 20, 40 from the open to the closed position as discussed herein. Potentially useful stapler actuation mechanisms may be described in, e.g., some of the stapler patents and patent publications identified herein.

Some illustrative methods of assembling the medical stapling apparatus of FIGS. 1 and 2 may include advancing the first stapling module 20 through the delivery lumen 12 in the delivery device 10 until the first stapling module 20 exits from the delivery lumen 12 at the distal opening 13. Advancing the first stapling module 20 may include advancing the first stapling module 20 along one or more guides 14, 16 passing through the guide lumens 30, 32 of the first stapling module 20.

The first stapling module 20 may preferably be oriented during the advancing with the second ends 23, 27 of the first and second jaws 22, 26 located ahead of the first ends 21, 25 of the first stapling module 20 such that the second ends 23, 27 of the first and second jaws 22, 26 exit the delivery lumen 12 before the second ends 23, 27 of the first and second jaws 22, 26.

The method may further include advancing the second stapling module 40 through the delivery lumen 12 until the second stapling module 40 exits from the delivery lumen 12 at the distal opening 13. In some embodiments, advancing the second stapling module 40 includes advancing the second stapling module 40 along one or more guides 14, 16 passing through the guide lumens of the second stapling module 40.

The method may further include aligning the first and second stapling modules 20, 40 with each other after at least the stapling module 40 has exited from the distal opening 13 of the delivery lumen 12. The aligning may include aligning the second ends 23, 27 of the first and second jaws 22, 26 of the first stapling module 20 with the second ends 43, 47 of the first and second jaws 42, 46 of the second stapling module 40 (see, e.g., FIG. 1).

When the stapling modules 20 and 40 are aligned as depicted in FIG. 1, the stapling modules 20 and 40 may be activated to move the jaws from the open position (FIG. 1) to the closed position (FIG. 2) as a part of the stapling process. In some embodiments, the guides 14 and 16 may be pulled taut to assist in alignment and/or operation of the stapling modules. In some embodiments, direct vision or imaging techniques may be used to verify positioning and/or alignment. In some embodiments, a cutter could be actuated in connection with the stapling. In some embodiments, removal of the stapling apparatus may be accomplished with the apparatus in its assembled form (see, e.g., FIG. 2) or, alternatively, the stapling apparatus could be completely or partially disassembled before removal.

In some embodiments, the first stapling module 20 and the second stapling module 40 may be advanced over one or more guides 14, 16. In some embodiments, advancing the first stapling module 20 includes pushing the first stapling module 20 through the delivery lumen 12 and pushing the second stapling module 40 through the delivery lumen 12.

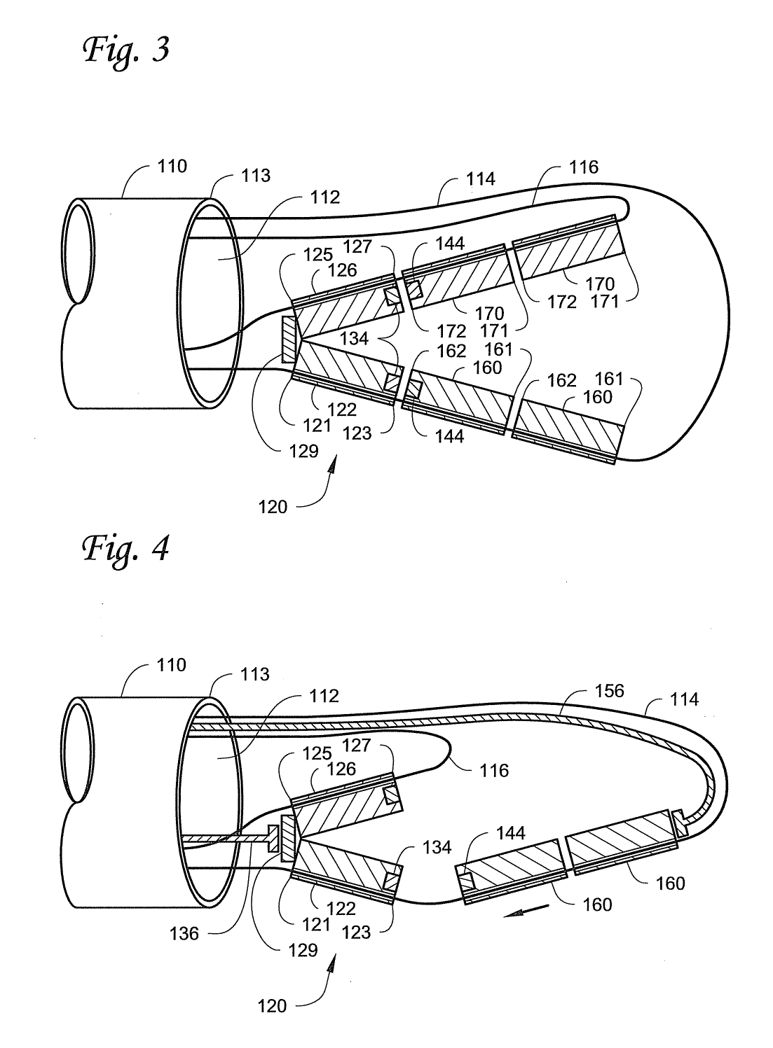

Another illustrative embodiment of a stapling apparatus and a method of assembling the stapler apparatus is depicted in FIGS. 3-5. The stapling apparatus of FIGS. 3-5 includes a base stapling module 120 that can be augmented by one or more supplemental stapling modules, each of which includes an anvil element 160 and an opposing staple driver 170. By adding one or more supplemental stapling modules to the base stapling module 120, the stapling apparatus can deliver staples over a larger operating distance than could be accomplished using the base stapling module 120 alone. Furthermore, the stapling apparatus can be assembled at a selected location after delivery of the different components through, e.g., an endoscope or other delivery device having a channel and one or more delivery lumens.

FIG. 3 depicts one embodiment of a stapling apparatus that includes a base stapling module 120 and two supplemental stapling modules, with each of the supplemental stapling modules including an anvil element 160 and a staple driver 170. FIG. 4 depicts an intermediate step in one embodiment of a method of assembling a stapling apparatus as depicted in FIG. 3, with the staple drivers 170 being advanced towards the base stapling module 120 along a guide 116.

The base stapling module 120 of the stapling apparatus of FIGS. 3 and 4 includes a first jaw 122 having a first end 121 and a second end 123, and a second jaw 126 having a first end 125 and a second end 127. The first end 125 of the second jaw 126 is pivotally attached to the first end 121 of the first jaw 122. In the depicted embodiment, the second jaw 126 may preferably include staple retaining structure adapted to retain one or more staples on the second jaw as discussed with the stapling apparatus described herein.

The base stapling module 120 depicted in FIGS. 3 and 4 also includes an actuation mechanism 129 (as described elsewhere herein) that is capable of selectively rotating the first jaw 122 and the second jaw 126 between an open position and a closed position. The second ends 123, 127 of the first jaw 122 and the second jaw 126 are closer to each other in the closed position than in the open position.

Although not depicted, the base stapling module 120 includes a guide lumen extending through the first jaw 122 of the base stapling module 120 and a guide lumen extending through the second jaw 126 of the base stapling module 120.

The stapling apparatus of FIGS. 3 and 4 is depicted as being delivered through a delivery device 110 that includes a delivery lumen 112 that extends through the delivery device 112, wherein the delivery lumen 112 terminates at a distal opening 113, and wherein the delivery lumen 112 is sized to allow passage of the base stapling module 120 through the delivery lumen 112.

The embodiment of the stapling apparatus depicted in FIGS. 3 and 4 also includes a guide 114 extending through the delivery device 110. The guide 114 passes through the guide lumen in the first jaw 122 of the base stapling module 120. The embodiment of the stapling apparatus depicted in FIGS. 3 and 4 also includes a guide 116 extending through the delivery device 110. The guide 116 passes through the guide lumen in the second jaw 126 of the base stapling module 120. The guides 114 and 116 may preferably be in the form of guidewires, although any structure (e.g., suture material, cables, ribbons, etc.) could be used in connection with the advancement and assembly of the stapling apparatus of FIGS. 3 and 4.

In addition to the base stapling module 120, the embodiment of the stapling apparatus depicted in FIGS. 3 and 4 preferably includes one or more supplemental stapling modules to extend the operating length of the stapling apparatus. In the depicted embodiment, each supplemental stapling module an anvil element 160 having a first end 161, a second end 162, and a guide lumen (not shown—but similar to the guide lumen depicted in connection with FIG. 1A) extending through the anvil element 160. In the depicted embodiment, the guide 114 passes through the guide lumen of the anvil element 160 of the supplemental stapling module.

Each supplemental stapling module of the depicted embodiment further includes a staple driver 170 having a first end 171, a second end 172, and a guide lumen (not shown—but similar to the guide lumen depicted in connection with FIG. 1A) extending through the staple driver 170. In the depicted embodiment, the guide 116 passes through the guide lumen of the staple driver 170. The staple driver 170 preferably includes staple retaining structure adapted to retain one or more staples on the staple driver 170 as discussed in connection with various embodiments of stapling apparatus described herein.

In some embodiments, the second end 123 of the first jaw 122 of the base stapling module 120 and the second end 162 of the anvil element 160 include complementary alignment elements 134, 144 to assist with alignment between the second ends 123, 162 of the first jaw 122 and the anvil element 160 when the second end 123 of the first jaw 122 faces the second end 162 of the anvil element 160. Similarly, the second end 127 of the second jaw 126 of the base stapling module 120 and the second end 172 of the staple driver 170 also include complementary alignment elements 134, 144 to assist with alignment between the second ends 127, 172 of the second jaw 126 and the staple driver 170 when the second end 127 of the second jaw 126 faces the second end 172 of the staple driver 170.

In some embodiments, the complementary alignment elements 134, 144 may be in the form of magnetic alignment elements that, through magnetic attraction and/or repulsion assist in aligning the second ends 123, 127 of the jaws 122, 126 of the base stapling module 120 with the second ends 162, 172 of the anvil element 160 and the staple driver 170 of a supplemental stapling module. In some embodiments, the complementary alignment elements 134, 144 may be in the form of mechanically interlocking structures, e.g., mating protrusions and recesses, keyed structures, etc. that may assist in aligning the second ends 123, 127 of the jaws 122, 126 of the base stapling module 120 with the second ends 162, 172 of the anvil element 160 and the staple driver 170 of a supplemental stapling module. In still other embodiments, both magnetic and mechanically interlocking structures may be used together to assist in alignment of the jaws of the base stapling module 120 with the anvil element 160 and the staple driver 170.

In some embodiments, the stapling apparatus of FIGS. 3 and 4 may also include a pushing element 136 capable of pushing the base stapling module 120 through the delivery lumen 112 of the delivery device 110. In some embodiments, a separate pushing element 156 may be provided to advance the anvil elements 160 and/or the staple drivers 170 along the guides 114 and 116. In still other embodiments, a dedicated pushing element may be used to advance the anvil elements 160 and a different dedicated pushing element may be provided to advance the staple drivers 170. In some embodiments, the pushing elements may be permanently or removably attached to the base stapling module 120, the anvil elements 160, and/or the staple drivers 170.

In some embodiments, the pushing elements may include a guide lumen through which guides 114, 116 may pass to assist in guiding the pushing elements during advancement of the base stapling module 120, the anvil elements 160, and/or the staple drivers 170.

In some embodiments, the pushing elements used in connection with the stapling apparatus of FIGS. 3 and 4 may have sufficient structure that they can be steered such that the direction and/or orientation of the base stapling module 120, the anvil elements 160, and/or the staple drivers 170. The use of steerable pushing elements may assist in assembling the supplemental stapling modules and/or in proper placement of the assembled stapling apparatus at a selected location.

In some embodiments, the guide lumen 164 in the anvil element 160 generally extends between the first end 161 and the second end 162 of the anvil element 160 such that movement of the anvil element 160 along the guide 114 aligns the anvil element 160 with the guide 114. In some embodiments, the guide lumen 174 in the staple driver 170 generally extends between the first end 171 and the second end 172 of the staple driver 170 such that movement of the staple driver 170 along the guide 116 aligns the staple driver 116 with the guide 116.

Assembly of the stapling apparatus may include, in some embodiments, advancing the base stapling module 120 through the delivery lumen 112 in the delivery device 110 until the base stapling module 120 exits from the delivery lumen 112 at the distal opening 113. The method may further include advancing one or more anvil elements 160 through the delivery lumen 112 and along the guide 114 that passes through the guide lumen 164 of the anvil element 160. The one or more anvil elements are advanced until the anvil elements 160 exit the delivery lumen 112 and the second end 162 of the anvil element 160 closest to the base stapling module 120 approaches the second end 123 of the first jaw 122 of the base stapling module 120. The method may further include aligning and operably attaching the second end 123 of the first jaw 122 to the second end 162 of the closest anvil element 160 after the anvil element 160 approaches the first jaw 122 as it advances along the guide 114.

The method may also include advancing one or more staple drivers 170 through the delivery lumen 112 and along the guide 116 passing through the guide lumen 174 of the staple driver 170. The one or more staple drivers 170 are advanced until the staple drivers 170 exit the delivery lumen 112 and the second end 172 of the staple driver 170 closest to the base stapling module 120 approaches the second end 127 of the second jaw 126 of the base stapling module 120. The method may further include aligning and operably attaching the second end 127 of the second jaw 126 to the second end 172 of the closest staple driver 170 after the staple driver 170 approaches the second jaw 126 as it advances along the guide 116.

In some embodiments, the method may include advancing the base stapling module 120 along the guides 114 and 116 as the base stapling module 120 is advanced through the delivery lumen 112 of the deliver device 110.

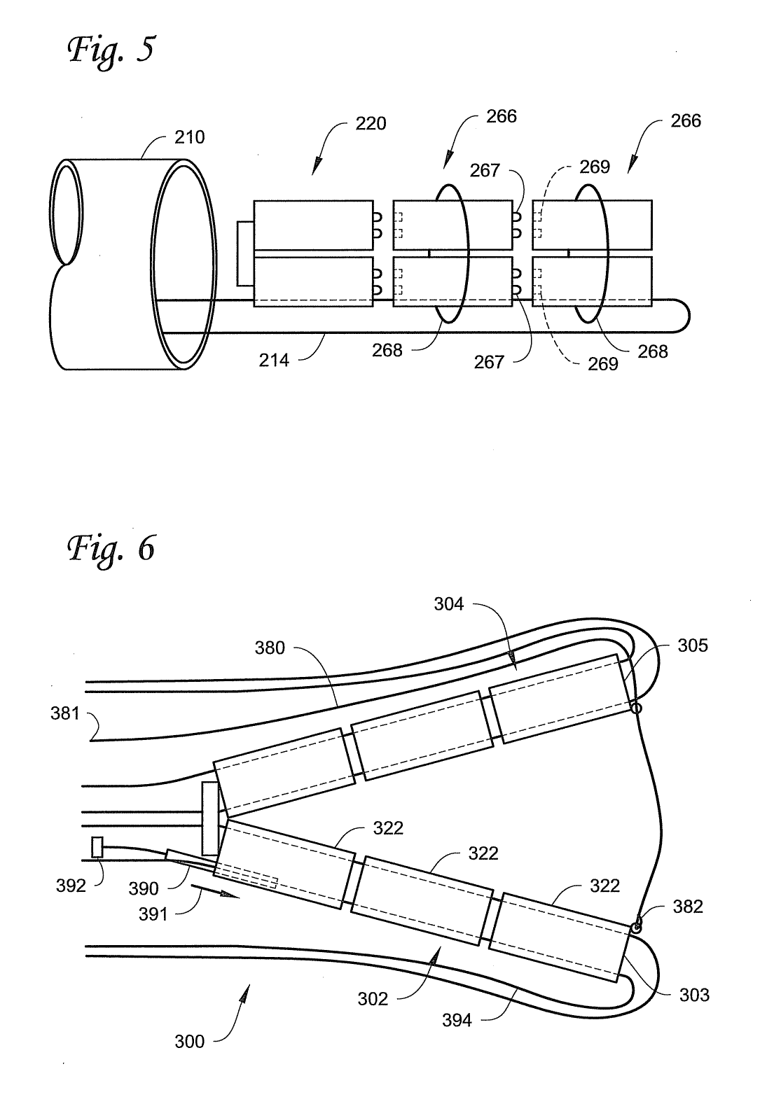

FIG. 5 depicts an optional feature that may be used in connection with some embodiments of the stapling apparatus described herein that may, in many respects, be similar to the stapling apparatus described above in connection with FIGS. 3 and 4. The stapling apparatus depicted in FIG. 5 includes a base stapling module 220 and two supplemental stapling modules 266 that each include an anvil element 260 and a staple driver 270. The stapling apparatus may be delivered through a delivery device 210.

The supplemental stapling modules 266 may be provided with a retaining structure 268 that holds the anvil elements 260 and the staple drivers 270 together while the stapling apparatus is being assembled. In such an embodiment, one potential advantage is that a single guide 214 may be used to advance the supplemental stapling modules 266 (rather than the two guides used in embodiments in which the anvil elements and the staple drivers are delivered independently of each other. In the illustrative embodiment of FIG. 5, the retaining structure 268 is in the form of a band, but many different retaining structures may be used, e.g., suture material, pins, water-soluble adhesives/materials, etc.

The base stapling module and the supplement stapling modules 266 also include one illustrative embodiment of mechanical interlocking structures that may be used to assist in alignment of the various components of the stapling apparatus. The mechanical interlocking structures depicted in FIG. 5 include posts 267 that mate with recesses 269, although a myriad of other interlocking structures could be used in place of the depicted structures.

Additional optional features that may be used with some embodiments of the stapling apparatus described herein are depicted in connection with FIG. 6. One of the optional features depicted in FIG. 6 is an alignment element 380 having a proximal end 381 and a distal end 382. The distal end 382 of the alignment element 382 is attached to the distal end 303 of jaw 302 of the stapling apparatus 300. It may be preferred that the distal end 382 of the alignment element 380 be fixedly attached to the first jaw 302 proximate the distal end 303 of the first jaw 302. The alignment element 380 extends from its attachment location on the first jaw 302 through an opening proximate the distal end 305 of the second jaw 326 before extending proximally towards the proximal end of the stapling apparatus 300.

The alignment element 380 may be in the form of a flexible member, e.g., a wire, cable, suture material, ribbon, etc. that can be placed in tension to assist in aligning the distal ends of the jaws of the stapling apparatus 300. Although not shown in connection with this embodiment, tensioning mechanisms are described herein and may be used to place the alignment element in tension between the distal ends 303, 305 of the jaws 302, 304 of the stapling apparatus 300.

In some embodiments, the alignment element 380 may also function to retain tissue located between the jaws 302 and 304 as the jaws 302, 304 are closed.

Another optional feature that may be used with some embodiments of the stapling apparatus described herein that is depicted in connection with FIG. 6 is a reinforcing pin 390 that may be advanced through a lumen until it is located such that is spans one or more of the junctions between the segments 322 of the jaw 302. The reinforcing pin 390 may be useful if, for example, additional rigidity needs to be imparted to the jaw 302 when it is constructed of two or more segments 322. In some embodiments, the reinforcing pin 390 may be advanced in the distal direction (see arrow 391) using a guide 394 threaded through a lumen or other structure in the segments 322. Although only one reinforcing pin 390 is depicted in FIG. 6, more than one pin could be used in the jaw 302. Furthermore, one or more reinforcing pins could be used in the jaw 304 if additional rigidity is desired there as well. Still further, the other stapling apparatus described herein that have a segmented jaw (see, e.g., FIG. 3) could also include one or more reinforcing pins if so desired.

Proper positioning of the reinforcing pin 390 relative to the junctions between the segments 322 may be achieved by a variety of different techniques. In the depicted embodiment, the reinforcing pin 390 is attached to a stop element 392 that is sized such that it cannot advance past a selected location, e.g., the opening in the first segment 322 into which the reinforcing pin 390 enters when moving in the distal direction. Many other arrangements for proper positioning of the pin 390 may be used in place of the depicted arrangement, e.g., the size of the lumen through which the reinforcing pin 390 passes may be reduced at a location such that the pin 390 cannot advance past the restriction, etc.

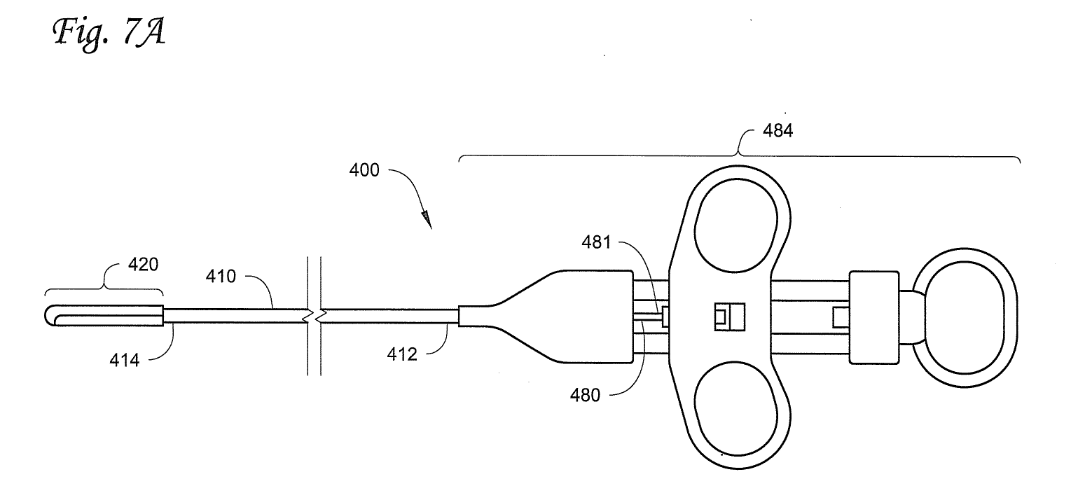

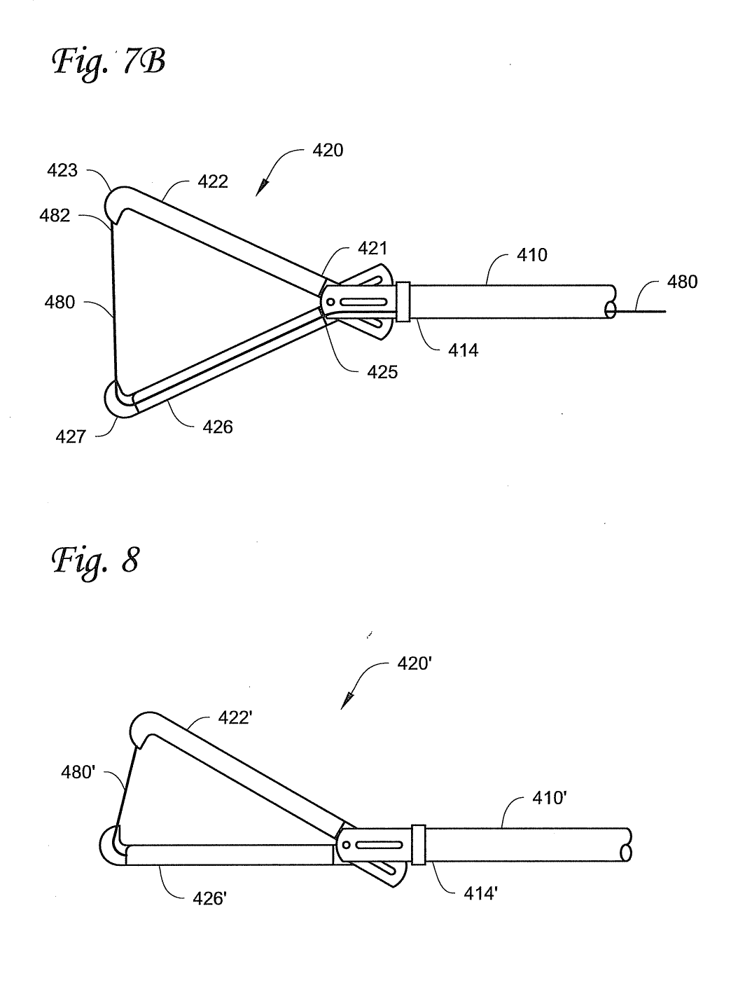

Yet another illustrative embodiment of a stapling apparatus is depicted in FIGS. 7A and 7B, where FIG. 7B is an enlarged side view of the distal portion of the stapling apparatus of FIG. 7A. The medical stapling apparatus of FIGS. 7A and 7B includes an elongated body 410 having a proximal end 412 and a distal end 414. A stapling module 420 is located at the distal end 414 of the elongated body 410.

The stapling module 420 includes a first jaw 422 having a proximal end 421 and a distal end 423, and a second jaw 426 having a proximal end 425 and a distal end 427. The proximal ends 421, 425 of the first jaw 422 and the second jaw 426 are pivotally attached to the distal end 414 of the elongated body 410 (although in some embodiments, such as that depicted in FIG. 8, only one of the jaws 422 and 426 may be pivotally attached to the distal end 414). The stapling module 420 further includes staple retaining structure adapted to retain one or more staples on at least one of the first jaw 422 and the second jaw 426.

The stapling apparatus of FIGS. 7A and 7B further includes a tension element 480 comprising a proximal end 481 and a distal end 482. The distal end 482 of the tension element 480 is fixedly attached to the first jaw 422 proximate the distal end 423 of the first jaw 422. The tension element 480 extends through an opening 483 proximate the distal end 427 of the second jaw 426 before extending proximally towards the proximal end 412 of the elongated body 410. The tension element 480 may be in the form of a flexible member, e.g., a wire, cable, suture material, ribbon, etc. that can be placed in tension to assist in closing the jaws 422 and 426 of the stapling module 420.

The stapling apparatus of FIGS. 7A and 7B includes one embodiment of a tensioning mechanism 484 proximate the proximal end 412 of the elongate body 410. The proximal end 481 of the tension element 480 is operably attached to the tensioning mechanism 484, wherein actuation of the tensioning mechanism 484 places the tension element 480 in tension between the distal ends 423, 427 of the first jaw 422 and the second jaw 426. That tension provides a force that can assist in closure of the jaws 422, 426. The tensioning mechanism 484 is, in the depicted embodiment, in the form of a slide 485 that can be moved proximally in the direction of arrow 486 to provide tension to the tension element 480. Many other tensioning mechanisms could be used in place of the depicted embodiment.

In some embodiments, the tension element 480 extends through a lumen in the first jaw 422 and/or through the elongated body 410, although in other embodiments, the tension element 480 may not be located within a lumen in the jaw 422 and/or the elongated body 410.

An alternative embodiment of a stapling apparatus 420′ similar to that depicted in FIG. 7B is depicted in FIG. 8. The depicted alternative embodiment includes a first jaw 422′ and a second jaw 426′. The first jaw 422′ is pivotally attached to the distal end 414′ of elongated body 410′ and the second jaw 426′ is stationary with respect to the distal end 414′. The pivotally-attached first jaw 422′ rotates relative to the distal end 414′ when the jaws 422′ and 426′ are closed. This embodiment of stapling apparatus 420′ also includes a tensioning element 480′ as described with respect to stapling apparatus 420 of FIGS. 7A and 7B.

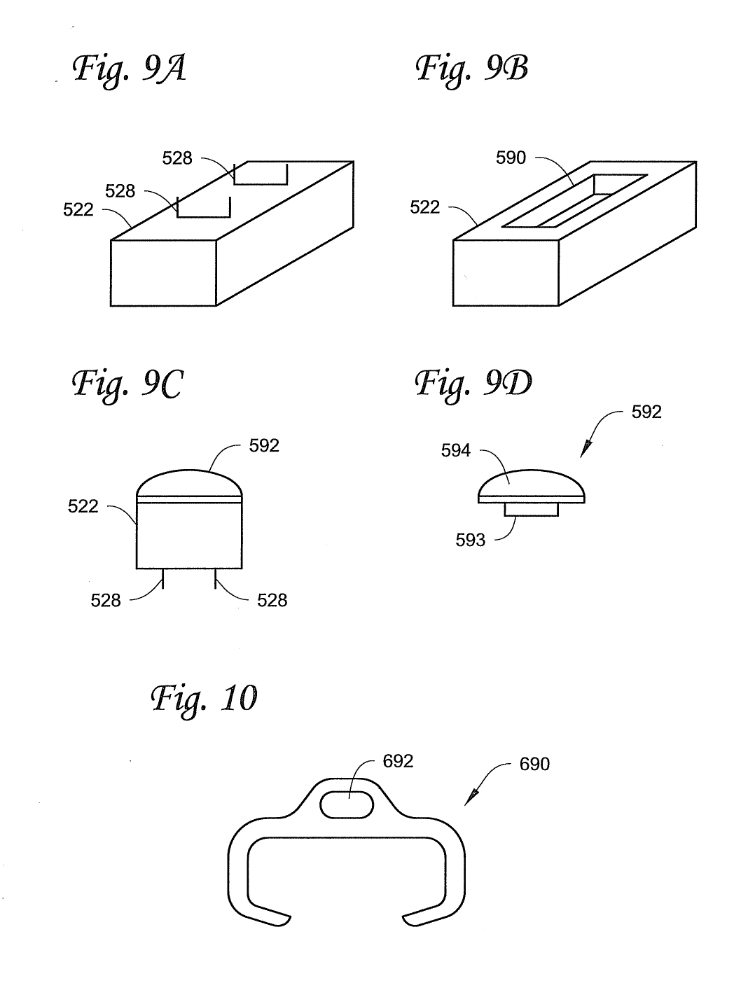

FIGS. 9A and 9B depict one illustrative embodiment of a stapler-delivered device that may be used in connection with the stapling apparatus described herein. The stapler-delivered device includes a body 522 that may, in some embodiments, be in the form of a jaw of one of the modular stapling apparatus described herein. The body 522 of the stapler-delivered device may include one or more staples 528 that may be used to attach the body 522 to tissue at a selected location.

The body 522 may, in various embodiments, have functional capabilities such as a providing a reservoir from which a drug or other agent may be delivered, housing a monitoring device capable of monitoring one or more physiological parameters such as, e.g., pH and/or other gastrointestinal parameters, cardiac activity, bacterial concentrations, etc. The drugs/agents that could be delivered from the body 522 may include, but are not limited to: chemotherapeutics, brachytherapy seeds, fiducial markers, anti-inflammatory drugs, antibiotics, immuno-suppressive drugs, prokinetics, antiemetics, etc.

In some embodiments, the body 522 may serve as a docking station to which other components may be attached such that they, along with the body 522, remain at a selected location because of the staples fixing the body 522 in position. The auxiliary components may, in some embodiments, be attached to the body 522 using, e.g., a mechanical structure such as, e.g., the slot 590 provided in body 522. Fixation of any auxiliary components to the body 522 may, however, be accomplished by any suitable technique or combination of techniques, e.g., a lock and key structure, threaded attachment, magnetic attachment, adhesives, etc.

Referring to FIGS. 9C and 9D, one illustrative embodiment of an auxiliary component 592 that may be attached to the body 522 includes a protrusion 593 that mates with the slot 590 in the body 522. The auxiliary component 592 may include, e.g., a reservoir 594 from which a drug/agent may be delivered (through elution, diffusion, pumping, etc.).

In some embodiments, one or both of the body 522 and any auxiliary component attached thereto (such as, e.g., auxiliary component 592) may include a power source (e.g., battery, etc.), communication components (e.g., a wireless transmitter and/or /transceiver such as, e.g., a Radio-Frequency ID device, etc.).

FIG. 10 depicts one illustrative embodiment of a staple adapted for use in attaching an auxiliary component as described above at a selected location in the absence of a separate body as described above. The depicted staple 690 includes an aperture 692 that can be used to attach an auxiliary component. Many other attachment features could be used in place of the aperture 692 in the depicted embodiment, e.g., a lock and key structure, threaded bore or post, magnetic materials, adhesives, etc.

Although not explicitly discussed herein, the stapling apparatus described herein may include cutting or resection elements where appropriate.

The stapling apparatus described herein may be constructed of any suitable materials that provide the properties required for the different structures and mechanisms, e.g., metals, polymers, ceramics, composites, etc.

The complete disclosure of the patents, patent documents, and publications cited herein are incorporated by reference in their entirety as if each were individually incorporated.

Illustrative embodiments of different stapling apparatus and methods of assembling or operating the same are discussed and reference has been made to possible variations within the scope of this invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

What is claimed is:

1. A medical stapling apparatus comprising:
a first stapling module and a second stapling module, wherein each of the first and second stapling modules comprises:
a first jaw comprising a first end and a second end;
a second jaw comprising a first end and a second end, wherein the first end of the second jaw is pivotally attached to the first end of the first jaw, wherein the second jaw comprises a staple retaining structure adapted to retain one or more staples on the second jaw;
an actuating mechanism capable of selectively rotating the first jaw and the second jaw between an open position and a closed position, wherein the second ends of the first jaw and the second jaw are closer to each other in the closed position than in the open position; and
a guide lumen extending through the first jaw and/or the second jaw of the stapling module.

2. An apparatus according to claim 1, wherein the second ends of the first and second jaws of the first and second stapling modules comprise complementary alignment elements such that the second ends of the first and second stapling modules align with each other when the second ends of the jaws of the first stapling module face the second ends of the jaws of the second stapling module.

3. An apparatus according to claim 2, wherein the complementary alignment elements comprise magnetic alignment elements.

4. An apparatus according to claim 2, wherein the complementary alignment elements comprise mechanically interlocking structures.

5. An apparatus according to claim 1, wherein the apparatus further comprises a pushing element capable of pushing the first stapling module and/or the second stapling module along a guide located in the guide lumen of the stapling module.

6. An apparatus according to claim 1, wherein the apparatus further comprises:
a first pushing element capable of pushing the first stapling module along a guide located in the guide lumen of the first stapling module; and
a second pushing element capable of pushing the second stapling module along a guide located in the guide lumen of the second stapling module.

7. An apparatus according to claim 1, wherein the guide lumen in the first and/or second stapling module generally extends between the first end and the second end of the jaw such that movement of the stapling module along a guide in the guide lumen aligns the jaw with the guide.

8. A method of assembling a medical stapling apparatus according to claim 1, the method comprising:
advancing the first stapling module through a delivery lumen in a delivery device until the first stapling module exits from the delivery lumen at a distal opening, wherein advancing the first stapling module further comprises advancing the first stapling module along a guide passing through the guide lumen of the first stapling module, and wherein the first stapling module is oriented during the advancing with the second ends of the first and second jaws located ahead of the first ends of the first stapling module such that the second ends of the first and second jaws exit the delivery lumen before the first ends of the first and second jaws;
advancing the second stapling module through the delivery lumen until the second stapling module exits from the delivery lumen at the distal opening, wherein advancing the second stapling module further comprises advancing the second stapling module along a guide passing through the guide lumen of the second stapling module; and
aligning the first and second stapling modules with each other after the first stapling module has exited from the distal opening of the delivery lumen, wherein the aligning comprises aligning the second ends of the first and second jaws of the first stapling module with the second ends of the first and second jaws of the second stapling module.

9. A method according to claim 8, wherein the first stapling module and the second stapling module are advanced over a guide.

10. A method according to claim 8, wherein aligning the first and second stapling modules comprises aligning magnetic alignment elements on the first and second stapling modules.

11. A method according to claim 8, wherein aligning the first and second stapling modules comprises aligning mechanically interlocking structures.

12. A method according to claim 8, wherein advancing the first stapling module comprises pushing the first stapling module through the delivery lumen.

13. A method according to claim 8, wherein advancing the first stapling module comprises pushing the first stapling module through the delivery lumen and wherein advancing the second stapling module comprises pushing the second stapling module through the delivery lumen.

14. A method according to claim 13, wherein pushing the first stapling module and the pushing the second stapling module are performed using the same pushing element.

15. A method according to claim 13, wherein pushing the first stapling module and the pushing the second stapling module are performed using two or more different pushing elements.

16. A medical stapling apparatus comprising:
a base stapling module that comprises:
a first jaw comprising a first end and a second end;
a second jaw comprising a first end and a second end, wherein the first end of the second jaw is pivotally attached to the first end of the first jaw, wherein the second jaw comprises a staple retaining structure adapted to retain one or more staples on the second jaw;
an actuation mechanism capable of selectively rotating the first jaw and the second jaw between an open position and a closed position, wherein the second ends of the first jaw and the second jaw are closer to each other in the closed position than in the open position;
a first guide lumen extending through the first jaw of the base stapling module; and
a second guide lumen extending through the second jaw of the base stapling module;
a delivery device comprising a delivery lumen that extends through the delivery device, wherein the delivery lumen terminates at a distal opening, and wherein the delivery lumen is sized to allow passage of the base stapling module through the delivery lumen;
a first guide extending through the delivery device, wherein the first guide passes through the first guide lumen in the first jaw of the base stapling module;
a second guide extending through the delivery device, wherein the second guide passes through the second guide lumen in the second jaw of the base stapling module; and
one or more supplemental stapling modules, wherein each supplemental stapling module comprises:
an anvil element comprising a first end, a second end, and a guide lumen extending through the anvil element, wherein the first guide passes through the guide lumen of the anvil element of the supplemental stapling module; and
a staple driver comprising a first end, a second end, and a guide lumen extending through the staple driver, wherein the second guide passes through the guide lumen of the staple driver, and wherein the staple driver comprises a staple retaining structure adapted to retain one or more staples on the staple driver.

17. An apparatus according to claim 16, wherein the second end of the first jaw of the base stapling module and the second end of the anvil element comprise complementary alignment elements such that the second ends of the first jaw and the anvil element align with each other when the second end of the first jaw faces the second end of the anvil element, and wherein the second end of the second jaw of the base stapling module and the second end of the staple driver comprise complementary alignment elements such that the second ends of the second jaw and the staple driver align with each other when the second end of the second jaw faces the second end of the staple driver.

18. An apparatus according to claim 17, wherein the complementary alignment elements comprise magnetic alignment elements.

19. An apparatus according to claim 17, wherein the complementary alignment elements comprise mechanically interlocking structures.

20. An apparatus according to claim 16, wherein the apparatus further comprises a pushing element capable of pushing the base stapling module through the delivery lumen of the delivery device.

21. An apparatus according to claim 16, wherein the apparatus further comprises:
a first pushing element capable of pushing the base stapling module through the delivery lumen of the delivery device;
a second pushing element capable of pushing the anvil element through the delivery lumen of the delivery device along the first guide; and
a third pushing element capable of pushing the staple driver through the delivery lumen of the delivery device along the second guide.

22. An apparatus according to claim 16, wherein the guide lumen in the anvil element generally extends between the first end and the second end of the anvil element such that movement of the anvil element along the first guide aligns the anvil element with the first guide, and wherein the guide lumen in the staple driver generally extends between the first end and the second end of the staple driver such that movement of the staple driver along the second guide aligns the staple driver with the second guide.

23. A method of assembling a medical stapling apparatus according to claim 16, the method comprising:
advancing the base stapling module through the delivery lumen in the delivery device until the base stapling module exits from the delivery lumen at the distal opening;
advancing the anvil element through the delivery lumen and along the first guide passing through the guide lumen of the anvil element, wherein the anvil element is advanced until the anvil element exits the delivery lumen and the second end of the anvil element approaches the second end of the first jaw of the base stapling module;
aligning and operably attaching the second end of the first jaw to the second end of the anvil element after the anvil element approaches the first jaw as it advances along the first guide;
advancing the staple driver through the delivery lumen and along the second guide passing through the guide lumen of the staple driver, wherein the staple driver is advanced until the staple driver exits the delivery lumen and the second end of the staple driver approaches the second end of the second jaw of the base stapling element; and
aligning and operably attaching the second end of the second jaw to the second end of the staple driver after the staple driver approaches the second jaw as it advances along the second guide.

24. A method according to claim 23, wherein advancing the base stapling module further comprises advancing the base stapling module along the first guide and the second guide.

25. A method according to claim 23, wherein aligning the first jaw and the anvil element comprises aligning a magnetic element on the first jaw with a complementary magnetic element on the anvil element.

26. A method according to claim 23, wherein aligning the second jaw of the base stapling module with the staple driver comprises aligning mechanically interlocking structures on the second jaw and the staple driver.

27. A method according to claim 23, wherein advancing the base stapling module comprises pushing the base stapling module through the delivery lumen.

28. A method according to claim 23, wherein advancing the anvil element comprises pushing the anvil element through the delivery lumen and wherein advancing the staple driver comprises pushing the staple driver through the delivery lumen.

29. A method according to claim 28, wherein pushing the anvil element and pushing the staple driver are performed using the same pushing element.

30. A method according to claim 28, wherein pushing the anvil element and pushing the staple driver are performed using two or more different pushing elements.

31. A medical stapling apparatus comprising:
an elongated body comprising a proximal end and a distal end;
a stapling module located at the distal end of the elongated body, wherein the stapling module comprises:
a first jaw comprising a proximal end and a distal end;
a second jaw comprising a proximal end and a distal end;
wherein the proximal end of at least one of the first jaw and the second jaw is pivotally attached to the distal end of the elongated body;
staple retaining structure adapted to retain one or more staples on at least one of the first jaw and the second jaw;
a tension element comprising a proximal end and a distal end, wherein the distal end of the tension element is fixedly attached to the first jaw proximate the distal end of the first jaw, and wherein the tension element extends through an opening proximate the distal end of the second jaw before extending proximally towards the proximal end of the elongated body; and
a tensioning mechanism proximate the proximal end of the elongate body, wherein the proximal end of the tension element is operably attached to the tensioning mechanism, wherein actuation of the tensioning mechanism places the tension element in tension between the distal ends of the first jaw and the second jaw.

32. An apparatus according to claim 31, wherein the tension element extends through a lumen in the first jaw.

33. An apparatus according to claim 31, wherein the tension element comprises a wire.

34. An apparatus according to claim 31, wherein both the first jaw and the second jaw are pivotally attached at their proximal ends to the elongated body.

35. A method of operating a medical stapling apparatus according to claim 31, the method comprising placing the tension element in tension between the distal ends of the first jaw and the second jaw, wherein the tension element moves through the opening in the second jaw when placing the tension element in tension.
